(19) United States
(12) Patent Application Publication
(10) Pub. No.: US 2007/0152568 A1
(43) Pub. Date: Jul. 5, 2007

(54) Compounds for an organic electroluminescent device and an organic electroluminescent device using the same

(76) Inventors: Chun-Liang Lai, Jiayi Hsien (TW); Kuo-Wei Huang, Xinzhuang City (TW)

Correspondence Address: Rabin & Berdo, P.C.;Suite 500, 1101 14th Street, Washington, DC, 20005, US

(21) Appl. No.: 11/320,001

(22) Filed: Dec. 29, 2005

(51) Int. Cl. H01L 51/54; C07C 211/43; C09K 11/06

(52) U.S. Cl. 313/504; 428/690; 428/917; 257/E51.049; 257/E51.051; 564/427; 564/434

(57) ABSTRACT

The present invention relates to a compound represented by formula (1), 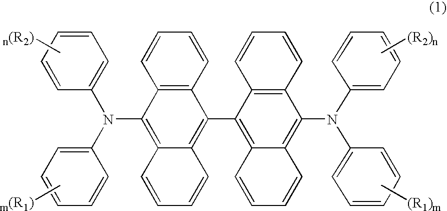
wherein each of R₁ and R₂, which may be identical or different, is selected from hydrogen, C₁-C₄ alkyl, C₁-C₄ alkoxy, C₁-C₄ alkylthio, unsubstituted C₆-C₁₂ aryl or C₆-C₁₂ aryl substituted by C₁-C₄ alkyl or C₁-C₄ alkoxy or C₁₂-C₂₀ diarylamino; and each of m and n is selected from an integer 0, 1, 2 or 3. The present invention also relates to an organic EL device using said compound as a dopant in at least one luminescent layer.

### BACKGROUND OF THE INVENTION

1. Field of the Invention

The present invention relates to compounds for an organic electroluminescent (EL) device and to an organic EL device using said compounds as dopants. The organic EL device in accordance with the present invention has high luminous efficiency and can emit light having wavelengths between 490˜580 nm.

2. Description of Prior Art

An organic EL device is typically composed of an anode, a cathode and layers of organic materials formed between the anode and the cathode. An organic EL device has many excellent properties such as a simple structure, low thickness, wide viewing angle, quick response, etc. Therefore, a lot of research and development related to organic EL devices has been conducted by many famous companies and research groups throughout the world. Currently, organic EL devices are widely used in display panels of MP3 players and sub-panels of cellular phones.

With improvements in manufacturing techniques and materials, the organic EL devices have made further progress in full-color developments. A lot of companies such as Kodak Company in the USA, Tohoku Pioneer Corporation and Hitachi, Ltd. in Japan, Samsung and LG in Korea and AU Optronics Corporation, CHI MEI Corporation and RiTdisplay Corporation in Taiwan have successively developed full-color displays using organic EL devices. Such a phenomena demonstrates how quickly organic EL devices have improved.

At least one of the layers formed between the anode and the cathode in an organic EL device is a luminescent layer. The at least one luminescent layer is composed of a host material and a dopant having high luminous efficiency. When a voltage is applied to the organic EL device, electrons and holes combine in the at least one luminescent layer, and the host material is excited and generates photons. Then, energy is transferred to the dopant by energy or charge transfer so the dopant is excited. When the dopant returns to the base state, the energy is released in the form of light. Therefore, the luminous efficiency and the color of light of an organic EL device depend on the dopant used. With the incorporation of a dopant into the host material, the energy can be utilized efficiently and will not transform to heat so the luminous efficiency of the organic EL device is superior to that employing a single luminescent material.

JP6009952 and JP7166160 disclose a coumarin derivative for use as a green light emitting material. The disadvantage of using the coumarin derivative as a dopant is that the range of doping concentration is small during evaporation and deposition. Kodak company made an improvement to overcome the above-mentioned disadvantage by using a di-(t-butyl)-substituted coumarin derivative to replace the coumarin derivative disclosed in U.S. Pat No. 6,020,078 and Appl. Phy. Lett., Vol. 79, No.22, P.3711. When the di-(t-butyl)-substituted coumarin derivative is used as a dopant, the range of doping concentration during evaporation and deposition increases, and the color of emitted light will not change with increasing currents. However, the di-(t-butyl)-substituted coumarin derivative is not highly heat-resistant, and its preparation is difficult.

Another green light emitting material disclosed in U.S. Pat. No. 5,593,788, U.S. Pat. No. 6,664,396 and TW200400778 is a quinacridone derivative. The quinacridone derivative has great heat-resistance. However, the solubility of this derivative is not desirable so testing the purity of obtained derivative is difficult.

JP2001131128 discloses a styryl derivative for use as a green light emitting material. The styryl derivative has a lot of double bonds so it has a disadvantage of bad heat-resistance. Further, JP2000268963 and JP2000268964 disclose a naphthacene derivative for use as a green light emitting material. However, the derivative does not have practical luminous efficiency and the cost for preparing said derivative is too high. Another green light emitting material disclosed in EP1403354 is a pyrene derivative. Nevertheless, this derivative also has the disadvantage of undesirable luminous efficiency.

EP 1246510, JP2002164175 and TW200527962 disclose arylaminoanthracene compounds for use as green light emitting materials in an organic EL device. While the color purity of the organic EL device has improved efficiently, the luminous efficiency of said organic EL device is not high enough (<12 cd/A).

Inventors of the present invention used a bianthracene based derivative in combination with a diarylamino substituent to obtain a diarylamino substituted bisanthracene derivative. The diarylamino substituted bisanthracene derivative was use as green light emitting dopant in an organic EL device that satisfies both the demands for high luminous efficiency and high color purity.

### SUMMARY OF THE INVENTION

An objective of the invention is to provide compounds represented by following formula (1), 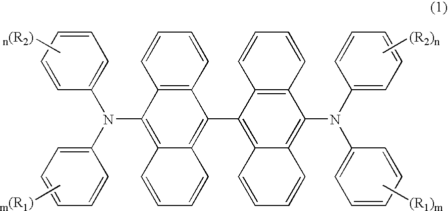
wherein each of R₁ and R₂, which may be identical or different, is selected from hydrogen, C₁-C₄ alkyl, C₁-C₄ alkoxy, C₁-C₄ alkylthio, unsubstituted C₆-C₁₂ aryl or C₆-C₁₂ aryl substituted by C₁-C₄ alkyl or C₁-C₄ alkoxy or C₁₂-C₂₀ diarylamino; and each of m and n is selected from an integer 0, 1, 2 or 3.

Another objective of the invention is to provide an organic EL device comprising a compound of formula (1) as a dopant. In addition, at least one luminescent layer of said organic EL device comprising a host material doped with a dopant of a compound of formula (1).

### DETAILED DESCRIPTION OF THE INVENTION

A compound in accordance with the present invention for use as a dopant in at least one luminescent layer of an organic EL device is represented by formula (1), 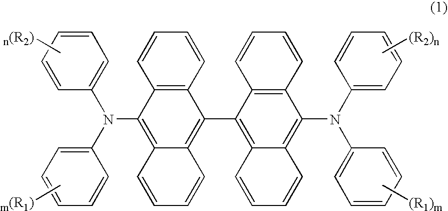
wherein each of R₁ and R₂, which may be identical or different, is selected from hydrogen, C₁-C₄ alkyl, C₁-C₄ alkoxy, C₁-C₄ alkylthio, unsubstituted C₆-C₁₂ aryl or C₆-C₁₂ aryl substituted by C₁-C₄ alkyl or C₁-C₄ alkoxy or C₁₂-C₂₀ diarylamino; and each of m and n is selected from an integer 0, 1, 2 or 3.

Examples of R₁ and R₂ in formula (1) include hydrogen, methyl, ethyl, propyl, isopropyl, butyl, isobutyl, tert-butyl, methoxy, ethoxy, tert-butyoxy, methylthio, phenyl, tolyl, naphthyl and diphenylamino.

In preferred compounds of formula (1), R₁ and R₂ independently of one another are methyl, tert-butyl or phenyl. Preferably, m and n independently of one another are an integer 0 or 1.

More preferably, the compound of formula (1) is, for example, one of the following compounds of formulas G1-G8:

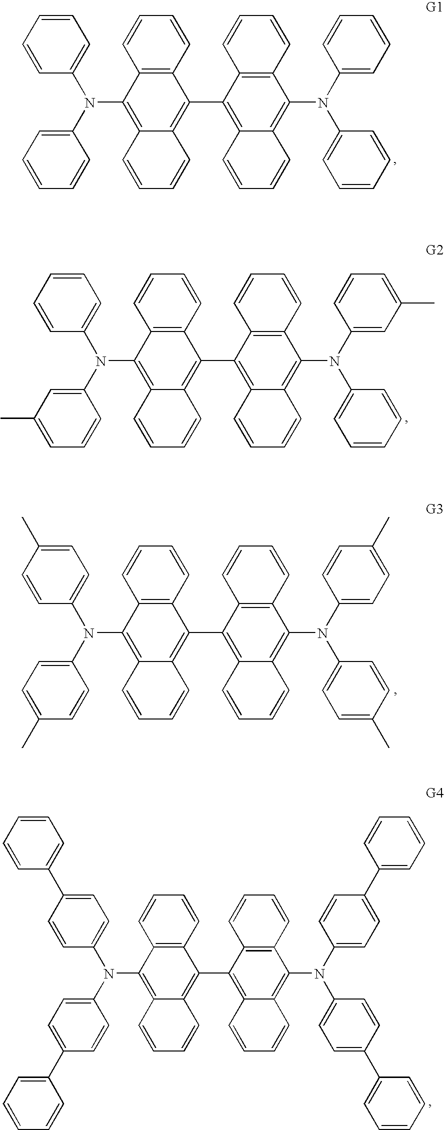

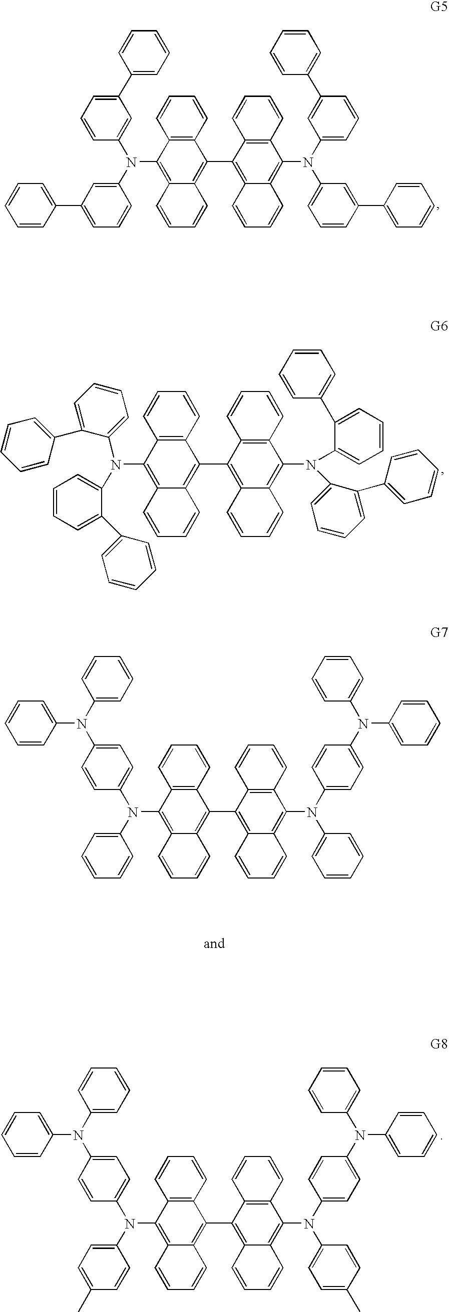

The compound of formula (1) can be prepared, for example, by known palladium-catalyzed reaction. For example, 10,10′-dibromo-[9,9′]bianthracene and diaryl substituted amine are reacted via coupling reaction with palladium as a catalyst to obtain the compound of formula (1). Alternatively, 10,10′-dibromo-[9,9′]bianthracene and aryl substituted primary amine are first reacted to obtain a secondary amine, and the obtained secondary amine is reacted with a halogen substituted aromatic compound to obtain the compound of formula (1).

The compounds of formula (1) obtained can be purified by column chromatography, recrystallization or sublimation, and the purity of the compounds can be above 99%. Sublimation is preferred for purification of the compounds since sublimation has the advantages of: 1) effectively removing mineral salts; 2) improving the particle compactness of the product; and 3) assuring completely drying the product to reduce factors causing degradation of an organic EL device.

An organic EL device may sequentially comprise:
(I) an anode, a hole-injecting layer, a hole-transporting layer, at least one luminescent layer, an electron-transporting layer, an electron-injecting layer and a cathode on a substrate,
(II) an anode, a hole-transporting layer, at least one luminescent layer, an electron-transporting layer, an electron-injecting layer and a cathode on a substrate, or
(III) an anode, a hole-transporting layer, at least one luminescent layer, an electron-transporting layer and a cathode on a substrate.
   The organic EL device comprising an anode, various layers and a cathode as described in (I) is preferred.

Typically, the manufacture of an organic EL device uses a substrate of a transparent material such as glass. Organic materials used in the formation of an organic EL device are heated in a vacuum (<10⁻³ torr) to 200˜600° C. to be directly vaporized in fabrication equipment and then subsequently deposited on the substrate to form films. The fabrication equipment uses a quartz vibrator to control the thickness of the films.

A conductive substance typically having a work function higher than 4 eV such as a metal, an alloy or the like is used as the anode. For example, the anode is made of indium-tin-oxide (ITO), gold or the like. The anode preferably has a resistivity of less than 199 Ω/□ and a thickness of 50˜200 nm.

The cathode is made of a conductive substance such as a metal, an alloy or the like and has a work function lower than 4 eV, e.g., Al, Li, Mg, Ag, Al—Li alloy, Mg—Ag alloy or the like. The cathode preferably has a thickness of 50˜200 nm.

The electron-injecting layer mainly consists of a metal or an inorganic ionic compound, such as LiF, CsF, Cs or the like. The electron-injecting layer preferably has a thickness of less than 1 nm.

The hole-injecting layer may be made from conventional phthalocyanine dyes, such as copper phthalocyanine, zinc phthalocyanine and the like; or triarylamine derivatives, such as m-TDATA (4,4′,4″-tris(N-3-methyl-phenyl-N-phenyl-amino) triphenylamine), 1-TNATA (4,4′,4″-tris(N-(1-naphthyl)-N-phenyl-amino) triphenylamine) and 2-TNATA (4,4′,4″-tris(N-(2-naphthyl)-N-phenyl-amino) triphenylamine); or p-phenylenediamine derivatives, such as N,N,N′,N′-tetra (2-naphthyl)-p-phenylenediamine, N,N,N′,N′-tetra(1-naphthyl)-p-phenylenediamine, N,N′-diphenyl-N,N′-di(4-(N″,N″-diphenylamino)phenyl)-biphenyldiamine, N,N′-di(1-naphthyl)-N,N′-di(4-(N″,N″-diphenylamino)phenyl)-biphenyldiamine and N,N′-diphenyl-N,N′-di(4-(N″-phenyl-N″-(1-naphthyl))phenylamino)-biphenyldiamine. The hole-injecting layer preferably has a thickness of 20˜80 nm.

The hole-transporting layer may be formed from conventional NPB (N,N′-di(naphthalene-1-yl)-N,N′-diphenyl-benzidine), PPB (N,N′-bis(phenanthren-9-yl)-N,N′-diphenylbenzidine), spiro-TAD (2,2′,7,7′-tetra-(diphenylamino)-9,9′-spiro-bifluorene) or N,N,N′,N′-tetra-(1-naphthyl)-biphenyldiamine. The hole-transporting layer preferably has a thickness of 10˜50 nm.

The at least one luminescent layer is composed of a host material and a dopant having high luminous efficiency. The highest occupied molecular orbital (HOMO) of the host material is is preferred to be lower than the HOMO of the dopant, and the lowest unoccupied molecular orbital (LUMO) of the host material is preferred to be higher than the LUMO of the dopant. In such combination of the host material and the dopant, occurrence of an exiplex can be prevented so energy can be transferred efficiently.

The compound of formula (1) is mainly used as the dopant in the at least one luminescent layer of an organic EL device. Suitable host materials for combination with the compound include but are not limited to metal complex, such as tris(8-hydroxyquinolinato)aluminum (Alq₃); anthracene derivatives, such as 9,10-di(2-naphthyl)anthracene, 2-methyl-9,10-di (2-naphthyl)anthracene, 2-tert-butyl-9,10-di(2-naphthyl)anthracene, 10,10-di (biphen-4-yl)-9,9′-bianthracene, 10,10-di(biphen-2-yl)-9,9-bianthracene, 10,10-di (2-naphthyl)-9,9-bianthracene; diphenylvinyl derivatives (2,2-diphenylvinyl derivatives), such as 4,4′-bis(2,2-diphenylvinyl)-1,1′-biphenyl (DPVBi) and 6,6′-bis(2,2′-diphenylvinyl)-2,2′-binaphthyl. Preferably, the host material is selected from anthracene derivatives.

Generally, the compound used as a dopant is preferably present in an amount of 0.5˜10% by weight of the host material. The luminescent layer preferably has a thickness of 10˜50 run. The maximum wavelength of the emitted light is between 490˜580 nm.

The electron-transporting layer of an organic EL device may be formed from a metal-quinolinate complex, such as Alq₃(tris(8-hydroxyquinolinato)aluminum), Bebq₂(bis(10-hydroxybenzo[h]quinolinato)beryllium), Gaq₃(tris(8-hydroxyquinolinato)gallium) and the like, a triazine derivative or an oxadiazole derivative. The metal-quinolinate complex is a commonly used electron-transporting material since it has high thermal stability and can be directly vaporized in a vacuum at elevated temperatures. The electron-transporting layer preferably has a thickness of 10˜50 nm.

An example of the fabrication of a preferred embodiment of the organic EL device according to the invention follows.

An anode is formed by deposition or sputtering of anode material by vacuum evaporation onto a suitable transparent substrate. Next, an electron-injecting layer, a hole-transporting layer, a luminescent layer, an electron-transporting layer and an electron-injecting layer are formed in sequence by deposition by vacuum evaporation. Generally, the vacuum is below 10⁻³ torr, and the rate of deposition is preferably 0.01˜5 nm/s. Finally, a cathode is formed by deposition or sputtering by vacuum evaporation to complete the organic EL device. The organic EL device may be suitably packaged and then can be operated in the atmosphere.

Alternatively, the organic EL device may be fabricated in a reversed sequence. Specifically, a cathode is first formed on the substrate and an electron-injecting layer, an electron-transporting layer, a luminescent layer, a hole-transporting layer and a hole-injecting layer are formed in sequence, and finally an anode is formed. When a direct current is applied, the organic EL device will emit light stably and continuously.

The following examples further illustrate the invention.

### EXAMPLES

### Example 1

### Synthesis of Compound G1 (10,10′-bis(N,N-diphenylamino)-9,9′-bianthracene)

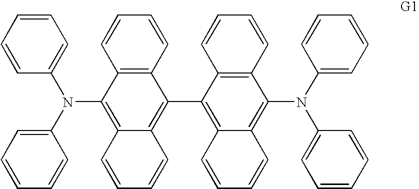

20 g of 10,10′-dibromo-[9,9′]-bianthracene, 15.8 g of diphenylamine, 9.0 g of tert-butyl sodium, 26 mg of palladium acetate and 80 mL of xylene were mixed in a reaction vessel and heated to 50° C. under a nitrogen atmosphere. 47 mg of tri(tert-butyl) phosphine was then added to the reaction vessel, and the reaction mixture was stirred continuously for 2 hours. 120 mL of methanol was added to the reaction vessel, and the reaction mixture was cooled to 25° C. The reaction mixture was filtered to obtain crude G1 product. The crude G1 product was purified by being recrystallized twice with methyl pyrrolidione, dried and sublimed to obtain 1 5.3 g of yellow solid G1 at a yield of 57%. The physical property of the compound G1 obtained is: Tm>320° C.

### Example 2

### Synthesis of Compound G2 (10,10′-bis(3-methyldiphenylamino)-9,9′-bianthracene)

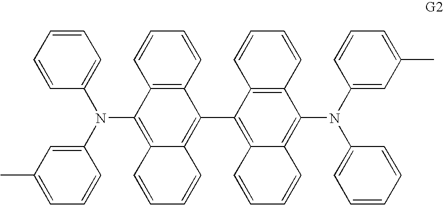

20 g of 10,10′-dibromo-[9,9′]-bianthracene, 17.2 g of di-p-toluidine, 9.0 g of tert-butyl sodium, 71 mg of tris(dibenzylideneacetone)dipalladium (Pd₂(dba)₃) and 78 mL of xylene were mixed in a reaction vessel and heated to 50° C. under a nitrogen atmosphere. 32 mg of tri(tert-butyl) phosphine was then added to the reaction vessel, and the reaction mixture was stirred continuously for 2 hours. 120 mL of methanol was added to the reaction vessel, and the reaction mixture was cooled to 25° C. The reaction mixture was filtered to obtain crude G2 product. The crude G2 product was purified by being recrystallized twice with methyl pyrrolidione, dried and sublimed to obtain 6.8 g of yellow solid G2 at a yield of 25% and purity of 99% (tested by HPLC with UV detector having set wavelength of 254 nm). The physical property of the compound G2 obtained is: Tm=320° C. and the photoluminescence (PL) maximum wavelength λmax=508 nm (in THF).

### Example 3

### Synthesis of Compound G3 (10,10′-bis(di-p-tolylamino)-9,9′-bianthracene)

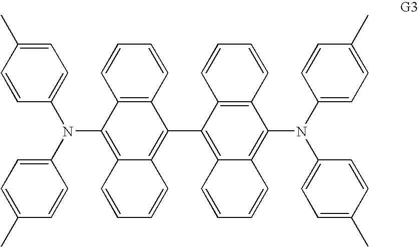

20 g of 10,10′-dibromo-[9,9′]-bianthracene, 18 g of di-p-toluidine, 9.0 g of tert-butyl sodium, 35 mg of palladium acetate and 80 mL of xylene were mixed in a reaction vessel and heated to 50° C. under a nitrogen atmosphere. 31 mg of tri(tert-butyl)phosphine was then added to the reaction vessel, and the reaction mixture was stirred continuously for 2 hours. 120 mL of methanol was added to the reaction vessel, and the reaction mixture was cooled to 25° C. The reaction mixture was filtered to obtain crude G3 product. The crude G3 product was purified by being recrystallized twice with methyl pyrrolidione, dried and sublimed to obtain 16 g of yellow solid G3 at a yield of 55% and purity of 99% (tested by HPLC with UV detector having set wavelength of 254 nm). The physical property of the compound G3 obtained is: Tm=395° C., UV−Vis λmax=565 nm (in THF) and PL λmax=508 nm (in THF).

### Example 4

### Synthesis of Compound G4 (N,N,N′,N′-tetra-p-biphenylyl-[9,9′]-bianthryl-10,10′-diamine)

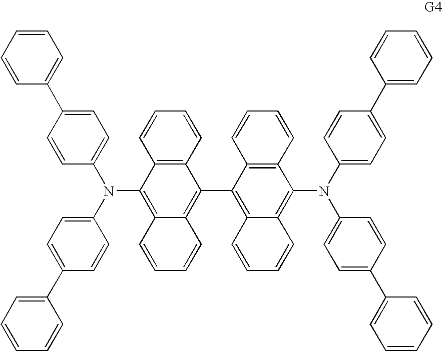

20 g of 10,10′-dibromo-[9,9′]-bianthracene, 30 g of di-p-toluidine, 9.0 g of tert-butyl sodium, 35 mg of palladium acetate and 80 mL of xylene were mixed in a reaction vessel and heated to 50° C. under a nitrogen atmosphere. 63 mg of tri(tert-butyl)phosphine was then added to the reaction vessel, and the reaction mixture was stirred continuously for 2 hours. 120 mL of methanol was added to the reaction vessel, and the reaction mixture was cooled to 25° C. The reaction mixture was filtered to obtain crude G4 product. The crude G4 product was purified by being recrystallized twice with methyl pyrrolidione, dried and sublimed to obtain 8.1 g of yellow solid G4 at a yield of 21% and purity of 99% (tested by HPLC with UV detector having set wavelength of 254 nm). The physical property of the compound G4 obtained is: Tm=428° C.

### Example 5

### Synthesis of Compound G7

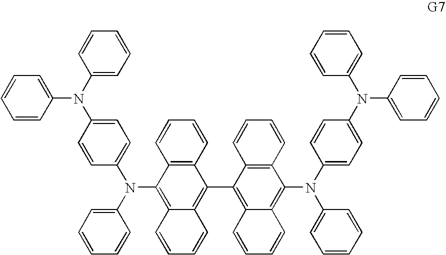 (a) synthesis of N,N′-diphenyl-[9,9′]-bianthryl-10,10′-diamine

30 g of 10,10′-dibromo-[9,9′]bianthracene, 16 g of phenylamine, 13.5 g of tert-butyl sodium, 214 mg of tris(dibenzylideneacetone)dipalladium (Pd₂(dba)₃) and 117 mL of toluene were mixed in a reaction vessel and heated to 50° C. under a nitrogen atmosphere. 94 mg of tri(tert-butyl)phosphine was then added to the reaction vessel, and the reaction mixture was stirred continuously for 2 hours. 180 mL of methanol was added to the reaction vessel, and the reaction mixture was cooled to 25° C. The mixture was filtered, and the solid obtained was dried at 120° C. to yield 29 g (yield 92% and purity 94%) of a yellow solid N,N′-diphenyl-[9,9′]-bianthryl-10,10′-diamine that was used in the next step without further purification.

(b) Synthesis of Compound G7

29 g of 10,10′-diphenyl-[9,9′]-bianthryl-10,10′-diamine obtained from (a), 35.5 g of 4-chloro triphenylamine, 12.5 g of tert-butyl sodium, 200 mg of tris(dibenzylideneacetone)dipalladium (Pd₂(dba)₃) and 108 mL of xylene were mixed in a reaction vessel and heated to 50° C. under a nitrogen atmosphere. 87 mg of tri(tert-butyl)phosphine was then added to the reaction vessel, and the reaction mixture was heated to 104° C. slowly and stirred continuously for 2 hours. 100 mL of methanol was added to the reaction vessel, and the reaction mixture was cooled to 25° C. The reaction mixture was filtered to obtain rough G7 product. The rough G7 product was purified by being recrystallized twice with methyl pyrrolidione, dried at 200° C. and sublimed to obtain 8.8 g of orange solid G7 at a yield of 16% and purity >99%. The physical property of the compound G7 obtained is: Tm=394° C.

### Example 6

### Fabrication of an Organic EL Device Using the Dopant According to the Invention

An ITO glass substrate used as an anode and having a surface resistivity of 20 Ω/□ was placed in a vapor deposition machine. A crucible containing N,N′-diphenyl-N,N′-di(4-(N″,N″-diphenylamino)phenyl)-biphenyldiamine (known as HI-01 and having the formula below), a crucible containing NPB, a crucible containing 2-tert-butyl-9,10-di(2-naphthyl)anthracene, a crucible containing compound G2 according to the present invention, a crucible containing tris(8-hydroxylquinolinato)aluminum (Alq₃), a crucible containing aluminum and a crucible containing lithium fluoride were put in the vapor deposition machine.

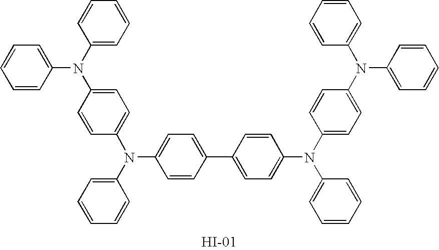

The pressure in the vacuum vessel of the vapor deposition machine was reduced to 10⁻⁶ torr. The crucible containing HI-01 was heated, and HI-01 was deposited on the glass substrate by evaporation at a rate of 0.2 nm/s to form a hole-injecting layer having a thickness of 60 nm. Next, a NPB film having a thickness of 40 nm was formed on the hole-injecting layer as a hole-transporting layer at a rate of 0.2 nm/s from the crucible containing NPB. Subsequently, the crucibles containing 2-tert-butyl-9,10-di(2-naphthyl)anthracene and compound G2 are heated, and a luminescent layer composed of 2-tert-butyl-9,10-di(2-naphthyl)anthracene incorporated with 3% of compound G2 was formed on the hole-transporting layer at a rate of 0.2 nm/s. The thickness of the luminescent layer is 30 nm. Then, an Alq₃ film having a thickness of 10 nm was formed on the luminescent layer as an electron-transporting layer from the crucible containing Alq₃. Thereafter, a lithium fluoride film having a thickness of 0.7 nm was formed on the electron-transporting layer as an electron-injecting layer by evaporation deposition from the crucible containing lithium fluoride. Finally, an aluminum cathode film having a thickness of 120 nm was formed on the electron-injecting layer from the crucible containing aluminum.

When a potential of 5.4V was applied to the organic EL device obtained, a green light was emitted with a light intensity of 8,475 cd/m², a luminance efficiency of 17 cd/A and a CIE coordinate of x=0.251, y=0.605.

### Example 7

### Fabrication of an Organic EL Device Using the Dopant According to the Invention

The procedure described in Example 6 was performed except that the luminescent layer was composed of 10,10-di(biphen-2-yl)-9,9-bianthracene incorporated with 4% of compound G2. When a potential of 10.7 V was applied to the organic EL device obtained, a green light was emitted with a light intensity of 21,279 cd/m², a luminance efficiency of 21.28 cd/A and a CIE coordinate of x=0.28, y=0.62.

### Example 8

### Fabrication of an Organic EL Device Using the Dopant According to the Invention

The procedure described in Example 6 was performed except that the luminescent layer was composed of 10,10-di(biphen-2-yl)-9,9-bianthracene incorporated with 8% of compound G2. When a potential of 11.53 V was applied to the organic EL device obtained, a green light was emitted with a light intensity of 21,326 cd/m², a luminance efficiency of 21.33 cd/A and a CIE coordinate of x=0.29, y=0.63.

### Comparison Example 1

### Fabrication of an Organic EL Device Using a Conventional Dopant

The procedure described in Example 6 was performed except that the luminescent layer was composed of tris(8-hydroxylquinolinato)aluminum incorporated with 1% of C-545T (10-(2-benzothiazolyl)-1,1,7,7,-tetramethyl-2,3,6,7-tetrahydro-1H,5H,11H-[1]benzopyrano[6,7,8-ij]quinolizin-11-one) having the formula shown below.

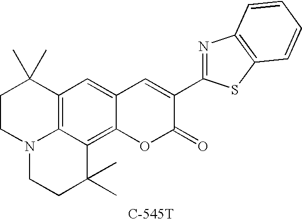

When a potential of 9 V was applied to the organic EL device obtained, a green light was emitted with a light intensity of 12,872 cd/m², a luminance efficiency of 14.98 cd/A and a CIE coordinate of x=0.263, y=0.656.

### Comparison Example 2

### Fabrication of an Organic EL Device Using a Conventional Dopant

The procedure described in Example 6 was performed except that the luminescent layer was composed of tris(8-hydroxylquinolinato)aluminum incorporated with 1% of tB-C-525T having the formula shown below.

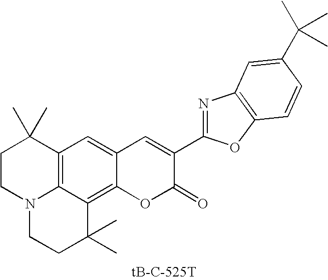

When a potential of 10 V was applied to the organic EL device obtained, a green light was emitted with a light intensity of 26,500 cd/M², a luminance efficiency of 10 cd/A and a CIE coordinate of x=0.28, y=0.59.

### Comparison Example 3

### Fabrication of an Organic EL Device Using a Conventional Dopant

The procedure described in Example 6 was performed except that the luminescent layer was composed of tris(8-hydroxylquinolinato)aluminum incorporated with 1% of DMQA having the formula shown below. 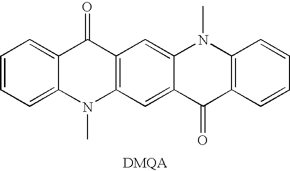
When a potential of 9 V was applied to the organic EL device obtained, a green light was emitted with a light intensity of 11,700 cd/m², a luminance efficiency of 13 cd/A and a CIE coordinate of x=0.30, y=0.62.

According to the data obtained in Examples 6, 7 and 8 as well as Comparison Examples 1 and 2, the luminous efficiency of the organic EL device is greatly improved when the luminescent layer in said organic EL device is doped with a compound of formula (1).

### INDUSTRIAL APPLICABILITY

When a compound of formula (1) is used as a dopant in combination with a host material to form at least one luminescent layer in an organic EL device, the device obtained has advantages of high luminous efficiency. Such an organic EL device can be advantageously used in display panels of MP3s, digital cameras, cellular telephones, etc.

What is claimed is:

1. A compound of formula (1), 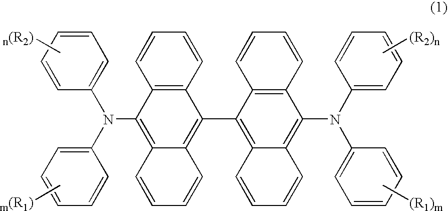
wherein each of R₁ and R₂, which may be identical or different, is selected from hydrogen, C₁-C₄ alkyl, C₁-C₄ alkoxy, C₁-C₄ alkylthio, unsubstituted C₆-C₁₂ aryl or C₆-C₁₂ aryl substituted by C₁-C₄ alkyl or C₁-C₄ alkoxy or C₁₂-C₂₀ diarylamino; and each of m and n is selected from an integer 0, 1, 2 or 3.

2. A compound according to claim 1 selected from a group consisting of:

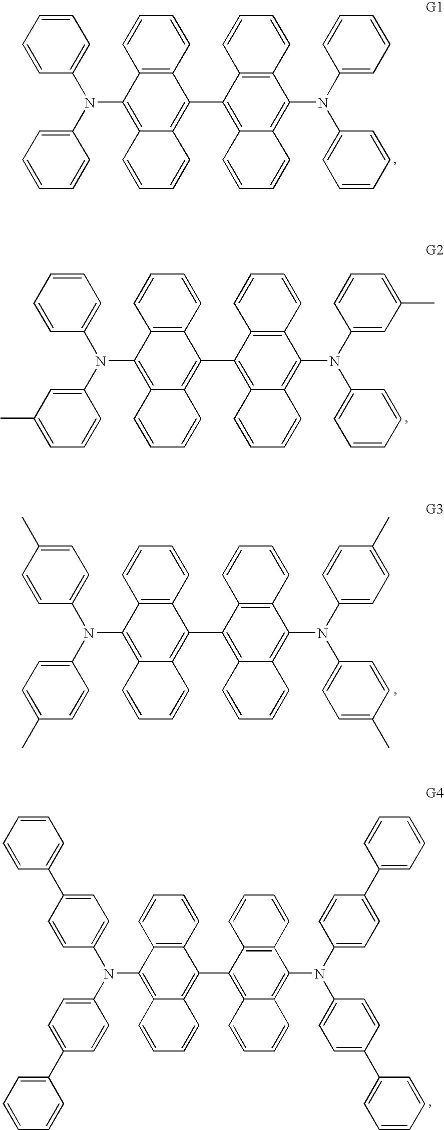

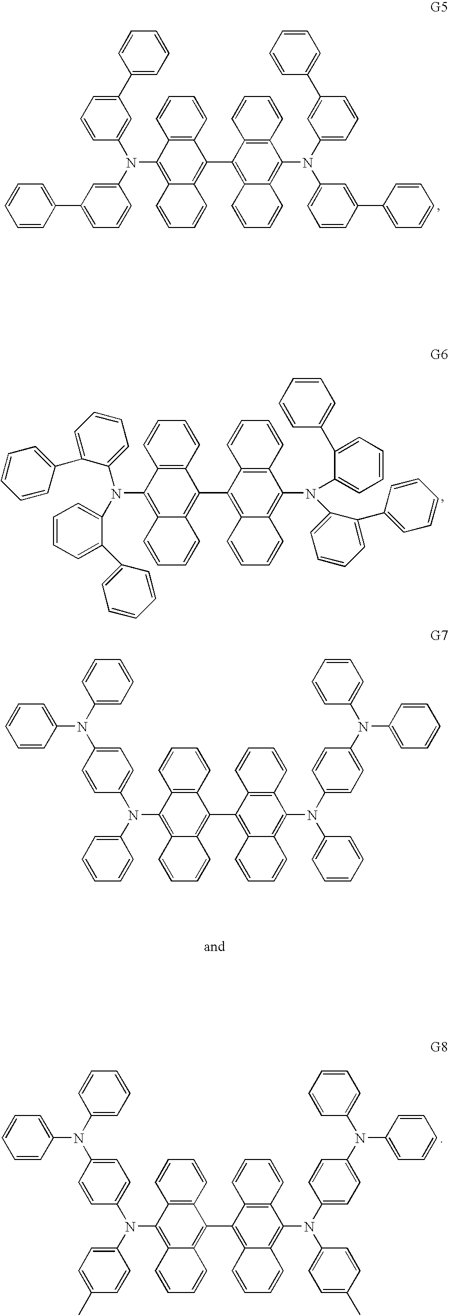

3. An organic EL device comprising at least one luminescent layer comprising a host material doped with a dopant of a compound of formula (1), 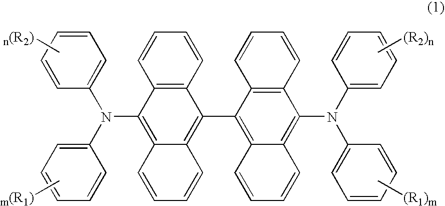
wherein each of R₁ and R₂, which may be identical or different, is selected from hydrogen, C₁-C₄ alkyl, C₁-C₄ alkoxy, C₁-C₄ alkylthio, unsubstituted C₆-C₁₂ aryl or C₆-C₁₂ aryl substituted by C₁-C₄ alkyl or C₁-C₄ alkoxy or C₁₂-C₂₀ diarylamino; and each of m and n is selected from an integer 0, 1, 2 or 3.

4. An organic EL device comprises at least one luminescent layer comprising a host material doped with a dopant of a compound selected from a group consisting of:

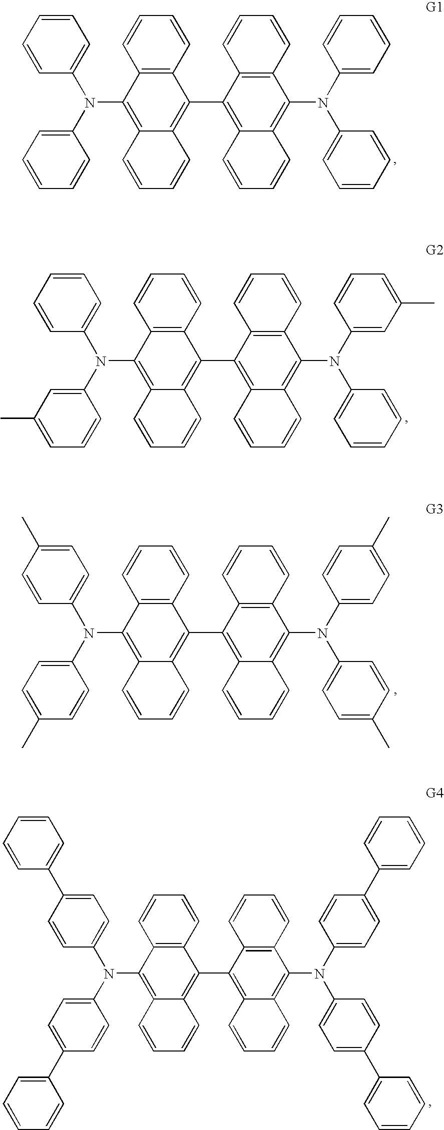

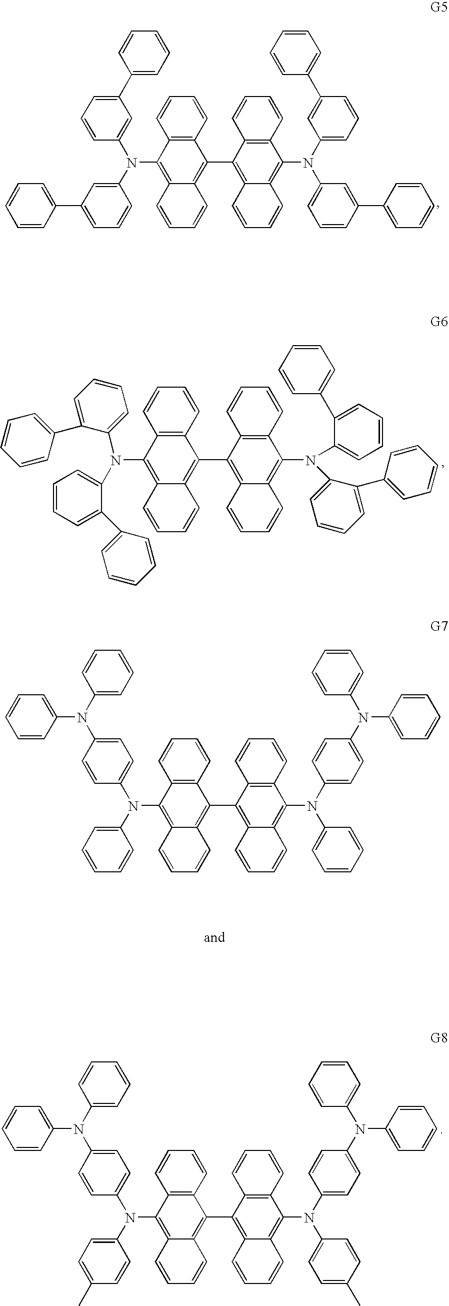

5. The organic EL device according to claim 3, wherein the dopant in the at least one luminescent layer is 0.5˜10% by weight of the host material.

6. The organic EL device according to claim 4, wherein the dopant in the at least one luminescent layer is 0.5˜10% by weight of the host material.

7. The organic EL device according to claim 3, wherein the host material is a metal complex, an anthracene derivative or a diphenylvinyl derivative.

8. The organic EL device according to claim 4, wherein the host material is a metal complex, an anthracene derivative or a diphenylvinyl derivative.

9. The organic EL device according to claim 7, wherein the host material is tris(8-hydroxyquinolinato)aluminum (Alq₃).

10. The organic EL device according to claim 7, wherein the host material is selected from the group consisting of 9,10-di(2-naphthyl)anthracene, 2-methyl-9,10-di(2-naphthyl)anthracene, 2-tert-butyl-9,10-di(2-naphthyl)anthracene, 10,10-di(biphen-4-yl)-9,9′-bianthracene, 10,10-di(biphen-2-yl)-9,9-bianthracene and 10,10-di(2-naphthyl)-9,9-bianthracene.

11. The organic EL device according to claim 7, wherein the host material is selected from the group consisting of 4,4′-bis(2,2-diphenylvinyl)-1,1′-biphenyl (DPVBi) and 6,6′-bis(2,2′-diphenylvinyl)-2,2′-binaphthyl.

12. The organic EL device according to claim 8, wherein the host material is tris(8-hydroxyquinolinato)aluminum (Alq₃).

13. The organic EL device according to claim 8, wherein the host material is selected from the group consisting of 9,10-di(2-naphthyl)anthracene, 2-methyl-9,10-di(2-naphthyl)anthracene, 2-tert-butyl-9,10-di(2-naphthyl)anthracene, 10,10-di(biphen-4-yl)-9,9′-bianthracene, 10,10-di(biphen-2-yl)-9,9-bianthracene and 10,10-di(2-naphthyl)-9,9-bianthracene.

14. The organic EL device according to claim 8, wherein the host material is selected from the group consisting of 4,4′-bis(2,2-diphenylvinyl)-1,1′-biphenyl (DPVBi) and 6,6′-bis(2,2′-diphenylvinyl)-2,2′-binaphthyl.
